(19) United States
(12) Patent Application Publication
(10) Pub. No.: US 2026/0038682 A1
(43) Pub. Date: Feb. 5, 2026

(54) TELEMEDICINE PLATFORM FOR LOCATION-BASED PROVIDER SCREENING AND REFERRAL

(71) Applicant: CheckMySpot, Inc., Florence, AL (US)

(72) Inventor: Joseph Michael Webb, Florence, AL (US)

(21) Appl. No.: 18/790,905

(22) Filed: Jul. 31, 2024

(51) Int. Cl. G16H 40/67; G16H 10/60; G16H 15/00; G16H 40/20

(52) U.S. Cl. CPC G16H 40/67; G16H 10/60; G16H 15/00; G16H 40/20

(57) ABSTRACT

Systems and methods are disclosed herein for a telemedicine platform for location-based provider screening and referral. The systems and methods can include a computing device that can receive patient data, such as patient biographical information and medical information, and image data, such as photographs of a spot on the patient's skin, from a patient computing device. The computing device can determine the geolocation of the patient computing device and assign the patient to a medical referral site based on the geolocation. The computing device can send a notification to a medical provider's computing device indicating that the patient's uploaded data is ready to be reviewed and provide access to the patient data. The provider can input case information via the provider's computing device to send to the computing device. The computing device can generate a case summary report with the patient data, image data, and case information and send the case summary report to the referral site.

### TECHNICAL FIELD

The present disclosure generally relates to telemedicine platforms, and more particularly to systems and methods for a telemedicine platform for location-based provider screening and referral.

### BACKGROUND

Skin cancer can be disfiguring and some forms, such as melanoma can be deadly. Time is of the essence when treating skin cancer, as early detection saves lives and improves outcomes. Asynchronous telemedicine (e.g., tele-dermatology), where communication between a patient and medical provider is not live, can aid in early detection. However, the utilization of tele-dermatology has been severely limited. For instance, federal regulations (e.g., Stark, HIPAA) impose requirements to avoid self-referral and require high levels of protection for patient historical information and identifiers. Additionally, most state regulations consider the practice of telemedicine to occur in the state where the recipient of care is physically located, which requires the provider of the telemedicine to be licensed to practice medicine in the state of the recipient. Most patients also do not have any formal education in medicine and are frequently confused by traditional medical terminology. This creates a situation in which patients become frustrated and dissatisfied with any telemedicine that does not assist their understanding by using simple terminology or development of an action plan that they can execute. What is needed is a platform and service that promotes the early detection of skin cancer, automatically generates a medical provider's results into a report that can be easily understood by a patient, and ensures that patients are referred to qualified medical providers in a location that is acceptable under federal and state laws and regulations.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure generally relates to telemedicine platforms, and more particularly to systems and methods for a telemedicine platform for location-based provider screening and referral. Asynchronous telemedicine, such as tele-dermatology, can aid in the early detection of disease in a patient, such as skin cancer. However, the utilization of tele-dermatology has been limited by laws and regulations that require patients to seek telemedicine in a location where they are physically located. The present disclosure provides a platform and service for asynchronous telemedicine (e.g., tele-dermatology) that allows patients and medical providers to participate in asynchronous telemedicine services in a proper location. Additionally, the platform can generate reports that categorize a patient's disposition into one of three categories, along with notes, provider information, and patient information, that are easy to understand. The platform can also automatically send the reports to a referral physician for patient follow-up in a location near the patient's physical location.

The platform can comprise a computing system with a central computing device (e.g., an administrative website or software) and user computing devices, such as computing devices for patients and medical providers with associated portals (i.e., user interfaces). The patient and provider computing devices can send and receive data to and from the central computing device, which can generate the reports based on the data and send the reports to the referral entity (e.g., a medical provider) that the central computing device determined was appropriate. For example, the central computing device can receive data from a patient (e.g., biographical information, contact information, medical history, symptoms, etc.) and a photograph of a skin lesion to be analyzed by a medical provider. The provider can be automatically assigned to the patient based on the physical locations of the patient and medical provider determined by using geolocation and based on one or more ratings, as non-limiting examples. The provider selected by the central computing device to participate in the asynchronous telemedicine session with the patient can receive a notification from the system that indicates that there is a case for the provider to review. The provider can use a computing device to provide case data (e.g., a case disposition or notes) to the central computing device. The central computing device can then use the patient data and the case data to generate a report that is color-coded and easy to understand and can send the report to a referral entity if the central computing device analyzes the data to determine that the provider recommended a referral. As a result, physicians in the proper geographical area can be quickly assigned to cases based on the precise geolocation, ensuring compliance with regulations. Additionally, the system can utilize HIPPA-compliant databases with both provider and referral physicians' contact, location, and availability information to be quickly accessed by the central computing device to determine whether a provider or referral is available within a set time period and to avoid a self-referral.

One aspect of the disclosure is a method. The method may include a method for location-based provider screening and referral. The method may include receiving, by a computing device, patient data and image data from a patient computing device associated with a patient. The method may include determining, by the computing device, a geolocation of the patient computing device. The method may include assigning, by the computing device, the patient to a referral entity based on the geolocation of the patient computing device. The method may include sending, by the computing device, the patient data and the image data to a provider computing device associated with a provider based on the geolocation of the patient computing device. The method may include receiving, by the computing device, case data from the provider computing device. The method may include generating, by the computing device, a report based at least in part on the patient data, the image data, and the case data. The method may include sending, by the computing device, the report to the referral entity based on the case data.

Another aspect of the disclosure is a non-transitory computer-readable storage medium having executable instructions stored thereon. The non-transitory computer-executable executable instructions, when executed by a processor, may be configured to perform one or more steps. The one or more steps may include receiving patient data and image data from a patient computing device associated with a patient. The steps may include determining a geolocation of the patient computing device. The steps may include assigning the patient to a referral entity based on the geolocation of the patient computing device. The steps may include sending the patient data and the image data to a provider computing device associated with a provider based on the geolocation of the patient computing device. The steps may include receiving case data from the provider computing device. The steps may include generating a report based at least in part on the patient data, the image data, and the case data. The steps may include sending the report to the referral entity based on the case data.

Another aspect of the disclosure is a system having at least one memory storing non-transitory computer-executable instructions stored thereon. The system may include a computing device with one or more processors for executing the non-transitory computer-executable instructions, a patient computing device with one or more input devices and associated with a patient, and a provider computing device associated with a provider. The non-transitory computer-executable instructions, when executed by the computing device, may cause the one or more processors of the computing device to perform one or more operations. The one or more operations may include receiving patient data and image data from the patient computing device. The operations may include determining a geolocation of the patient computing device. The operations may include assigning the patient to a referral entity based on the geolocation of the patient computing device. The operations may include providing, based on the geolocation of the patient computing device, the provider computing device with access to the patient data and the image data. The operations may include receiving case data from the provider computing device. The operations may include generating a report based at least in part on the patient data, the image data, and the case data. The operations may include sending the report to the referral entity based on the case data.

Numerous other objects, advantages and features of the present disclosure will be readily apparent to those of skill in the art upon a review of the following drawings and description of various embodiments.

### BRIEF DESCRIPTION OF THE DRAWINGS

The accompanying drawings, which are incorporated in and constitute a part of the specification, illustrate exemplary embodiments of the disclosure. Together with the description, they serve to explain the objects, advantages, and principles of the disclosure.

FIG. 1 is a block diagram illustrating an example embodiment of a system of the present disclosure.

FIGS. 2A-2F are front views illustrating example embodiments of a patient interface of a patient computing device of the system of the present disclosure.

FIGS. 3A-3E are front views illustrating example embodiments of case summary reports generated by the system.

FIG. 4 is a flowchart diagram illustrating an example embodiment of a method of the present disclosure.

### DETAILED DESCRIPTION

Reference will now be made in detail to exemplary embodiments of the disclosure, some aspects of which are illustrated in the accompanying drawings.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

Multiple elements of the same or a similar type may be referred to as “Elements 102(1)-(n)” where n may include a number. Referring to one of the elements as “Element 102” refers to any single element of the Elements 102(1)-(n). Additionally, referring to different elements “First Elements 102(1)-(n)” and “Second Elements 104(1)-(n)” does not necessarily mean that there must be the same number of First Elements as Second Elements and is equivalent to “First Elements 102(1)-(n)” and “Second Elements (1)-(m)” where m is a number that may be the same or may be a different number than n.

The present disclosure is directed to systems and methods for a telemedicine platform for location-based provider screening and referral. A platform may provide a patient interface for a patient to upload data about the patient and a spot on the skin which the patient needs examined. The platform may provide a provider interface for a medical provider (e.g., a qualified dermatologist) to review the patient's uploaded data and provide case data including a case disposition. The platform may be integrated within a computing system that generates the patient and provider interfaces, uses geolocation to select the provider based on the location of the patient's computing device from which the patient uploaded data via the patient interface, uses the case data and patient data to generate a case summary report, and sends the case summary report to a referral entity (e.g., a qualified dermatologist different from the provider) based on the geolocation. This system allows physicians in the proper geographical area to be quickly assigned to cases based on the precise geolocation, ensuring compliance with regulations, and uses both provider and referral physicians' location and availability information to determine whether a provider or referral is available and to avoid a self-referral.

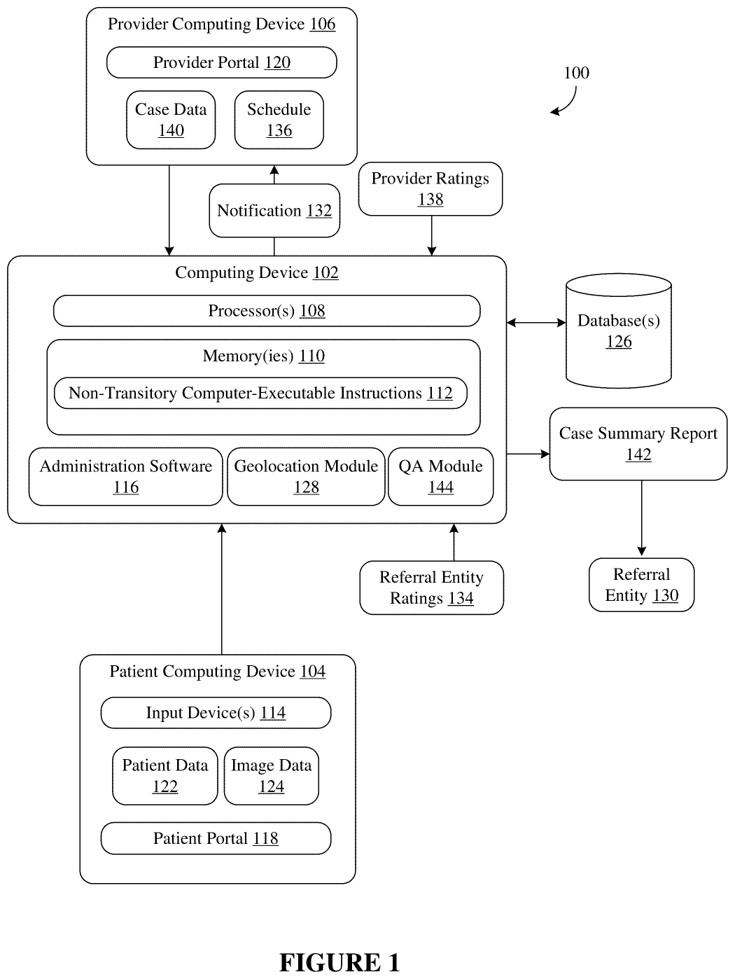

FIG. 1 is a block diagram illustrating an example embodiment of a system 100 of the present disclosure. The system 100 can implement the telemedicine platform for location-based provider screening and referral. The system 100 may include a combination of software and hardware. The system 100 may include one or more computing devices (e.g., computing device 102, patient computing device 104, provider computing device 106), such as desktop computers, laptop computers, mobile computing devices (e.g., a mobile phone or tablet computer), mobile medical carts with a computer installed on the cart, application servers, database servers, and other computing devices.

The system 100 may include a computing device 102, such as a central computing device, cloud, or server, one or more patient computing devices 104, and one or more provider computing devices 106. The patient computing device 104 may be associated with a patient seeking telemedicine services, and the provider computing device 106 may be associated with a qualified medical provider that can provide the patient with the requested telemedicine services. The computing device 102 may be in data communication with the patient computing device 104 and the provider computing device 106 over a data network (e.g., the internet, a local area network (LAN), or a wide area network (WAN)).

The computing device 102 may include at least one processor 108. The computing device 102 may include one or more memories 110. The memories 110 can store non-transitory computer-executable instructions 112. The patient computing device 104 and the provider computing device 106 can bothv include at least one processor 108 and one or more memories 110 that can store non-transitory computer-executable instructions 112. The patient computing device 104 may include one or more input devices 114, such as cameras, microphones, and text input devices, as non-limiting examples. For instance, the input devices 114 may include a camera that takes photographs (e.g., image data 124). The image data 124 may be stored in a memory 110 or storage device of the patient computing device 104. The storage device may include a hard drive disk, a solid-state drive, a memory (such as random-access memory (RAM), a flash memory, or some other type of memory), as non-limiting examples. The system 100 can be configured to utilize health industry-standard security protocols at all points of data transmission to safeguard patient data and privacy, such as TLS (1.1 or above) encrypted and/or HIPAA (Health Insurance Portability and Accountability Act) compliant storage, as non-limiting examples.

The computing device 102 may include software that facilitates interaction with the patient computing device 104 and the provider computing device 106, such as administration software 116. For instance, the software (e.g., administration software 116) may facilitate the generation and display of a patient portal 118 and a provider portal 120 that allows the patient and provider, respectively, to interact with the system 100. The patient portal 118 can be provided for the patient-patient associated with the patient computing device 104 to allow the patient to upload data, such as biographical information, medical history, symptoms, and photographs of a spot of concern (e.g., a skin lesion), as non-limiting examples. For example, the patient portal 118 may be provided via an application downloaded to the patient computing device 104. The provider portal 120 can be presented for the medical provider associated with the provider computing device 106 to allow the provider to receive data, such as the patient data 122 that was uploaded, and to upload data, such as biographical information about the provider, comments, notes, and case disposition, as non-limiting examples. The software (e.g., administration software 116) may enable the patient portal 118 and the provider portal 120 by providing a secure website specifically for the patient (the patient portal 118) and a secure website specifically for the medical provider (the provider portal 120). For instance, HIPAA-compliant servers can be utilized by the software to provide secure websites for the patient and medical provider to ensure that any patient data 122 (i.e., protected health information) is properly protected.

The non-transitory computer-executable instructions 112, upon execution, can cause the computing device 102 to perform one or more of the operations or methods described herein. The computing device 102 may receive patient data 122 and image data 124 from the patient computing device 104 associated with a patient. Prior to receiving the patient data 122 and the image data 124 from the patient computing device 104, the patient may create an account with the platform (i.e., the system 100) by entering information, such as the patient's name, phone number, date of birth, email, and password, as non-limiting examples. If the patient has an account, the patient may enter their login information (e.g., email and password) into the patient portal 118, for example, to be authenticated by the system 100 (e.g., the administration software 116) and log the patient computing device 104 into the system 100 (e.g., via the patient portal 118). The information that the patient entered may comprise the patient data 122. Thus, the patient data 122 can include the patient's name, phone number, date of birth, email, and password. Additionally, the patient data 122 can include information about the patient's condition that is to be examined by the medical provider, such as a medical history and symptoms, as non-limiting examples, which can be uploaded by the patient in response to being prompted by the system 100 to enter such information via the patient portal 118. For instance, the patient data 122 may allow the system 100 to create a patient profile for the patient and store the profile in a HIPAA-compliant database 126. The image data 124 can include one or more photographs of a spot on the patient's skin the patient wants examined by a medical provider, which the patient took photos of using a camera of the patient computing device 104 and uploaded via the patient portal 118.

In some embodiments, the system 100 can provide the patient computing device 104 with access to a video to view regarding the proper uploading of photographs taken by the patient computing device 104. The patient data 122 and the image data 124 can be automatically sent to a database 126 that is HIPAA compliant. The database 126 can be configured to store and retrieve information associated with the system 100. For example, the patient data 122 and the image data 124 can be received by the computing device 102 when the computing device 102 accesses the database 126 to retrieve the patient data 122 and the image data 124, or by a HIPAA-compliant, secure communication of the patient data 122 and the image data 124 from the patient computing device 104 to the computing device 102.

As used herein, “patient” may include a patient seeking medical diagnosis via asynchronous telemedicine services or a representative interacting with the system on behalf of a patient seeking medical diagnosis via asynchronous telemedicine services unless stated otherwise or as dictated by the context. For example, in the context of a patient taking an action such as using the patient computing device 104 or the patient portal 118, references to “patient” could typically include a user interacting with the system 100 on behalf of the patient. However, in the context of patient data 122, patient symptoms, patient conditions, patient medical history, the geolocation 202 of the patient computing device 104, or other similar contexts, references to “patient” are generally limited only to the patient and not a user interacting with the system 100 on behalf of the patient.

The computing device 102 may determine a geolocation 202 of the patient computing device 104. For example, the computing device 102 may include a geolocation module 128 that uses geolocation algorithms to determine an accurate location (e.g., coordinates) of the patient computing device 104. Determining the geolocation 202 of the patient computing device 104 ensures that a medical provider that will be reviewing the patient data 122 and the image data 124 is licensed in the same state as the patient, as many state medical boards and regulations provide that the practice of medicine is to occur at the physical site of the recipient. Determining the geolocation 202 of the patient computing device 104 also ensures that a referral entity 130 that the patient may be referred to is located nearby to the user (e.g., within a particular predetermined distance, such as 1 mile, 5 miles, 10 miles, 25 miles, the same state, etc.).

The computing device 102 (e.g., the administration software 116) can assign the patient to a referral entity 130 based on the geolocation 202 of the patient computing device 104. The computing device 102 (e.g., the administration software 116) can also assign the patient to a provider associated with a provider computing device 106 based on the geolocation 202 of the patient computing device 104. The computing device 102 can provide a payment selection screen to the patient computing device 104 to receive payment for the services from the patient, such as via a third-party payment provider.

In some embodiments, to assign the patient to the referral entity 130 based on the geolocation 202 of the patient computing device 104, the computing device 102 may use the geolocation 202 of the patient computing device 104 to identify one or more referral entities 130 that are located within a particular predetermined distance (e.g., 1 mile, 5 miles, 10 miles, 25 miles, the same state, etc.) from the geolocation 202 of the patient computing device 104. For instance, the database 126 may include information about qualified referral entities 130 (e.g., addresses and/or precise location coordinates) for the computing device 102 to choose from and the computing device 102 may identify which of these qualified referral entities 130 are within the predetermined distance by accessing the database 126 and determining the distances between the referral entities 130 and the geolocation 202 of the patient computing device 104. The computing device 102 may receive ratings 134 for each of the referral entities 130 located within the predetermined distance, such as a “Referral Site Quality Rating” that may be based upon the timeliness of referrals receiving appointments in less than one week, by accessing a database 126 or other component of the system 100 to obtain the ratings 134. The computing device 102 may select the referral entity 130 based on the ratings 134, such as by selecting the highest rated of the referral entities 130. In some examples, the computing device 102 may select the referral entity 130 by selecting from a subset of the referral entities 130, such as the top 3 highest rated, the top 5 highest rated, the top 10 highest rated, etc. In some examples, the computing device 102 may monitor the availability or wait times of referral entities 130 within the predetermined distance from the geolocation 202 of the patient computing device 104 and assign the patient to a referral entity 130 that has the soonest availability (e.g., when the case disposition is “worrisome”).

In some embodiments, to assign the patient to the referral entity 130 based on the geolocation 202 of the patient computing device 104, the computing device 102 may determine that the patient selected a preferred referral entity 130 and that the preferred referral entity 130 is one of the one or more referral entities 130 that are located within the predetermined distance. For example, the patient may have a physician they prefer to visit if a referral is needed and enter information about the physician via the patient portal 118 for the computing device 102 (e.g., the administration software 116) to receive and use when assigning the patient to the referral entity 130. The computing device 102 may obtain information associated with the preferred referral entity 130 from a database 126 configured to store information associated with the referral entities 130, such as provider names and contact information, as non-limiting examples. The computing device 102 can use such information to communicate with the preferred referral entity 130 if a referral is determined to be needed (e.g., to send the case summary report 142). In some examples, the computing device 102 may determine that there is no entry for the preferred referral entity 130 in the database 126 and add an entry to the database 126 with the information associated with the preferred referral entity 130, such as provider names and contact information, as non-limiting examples.

In some embodiments, the computing device 102 may use the geolocation 202 of the patient computing device 104 to identify one or more providers that are located within a particular predetermined distance (e.g., 1 mile, 5 miles, 10 miles, 25 miles, the same state, etc.) from the geolocation 202 of the patient computing device 104. The computing device 102 (e.g., the administration software 116) may receive a schedule 136 from each of the providers via the provider portal 120 of each of the provider computing devices 106 that represents the availability and unavailability of each provider. The schedules 136 may be received from the provider computing devices 106 at a time prior to the computing device 102 identifying the providers based on the geolocation 202 of the patient computing device 104, and the computing device 102 may access the schedules 136 of only the providers that are within the predetermined distance. The computing device 102 may receive provider ratings 138 for each of the providers located within the predetermined distance, such as a provider quality assurance rating based on timeliness and/or adherence to a quality assurance review, by accessing a database 126 or other component of the system 100 to obtain the provider ratings 138. The computing device 102 may select the provider for the patient based on the provider ratings 138 and based on the schedule 136 of each of the providers, such as by selecting the highest rated of the providers that are available within the next 24-48 hours. In some examples, the computing device 102 may select the provider for the patient by selecting from a subset of the providers, such as the top 3 highest rated, the top 5 highest rated, the top 10 highest rated, etc., that are also available within the next 24-48 hours. The provider computing device 106 can be associated with the provider selected by the computing device 102. The provider selected by the computing device 102 is different from the referral entity 130 to avoid self-referral, so the computing device 102 may compare the selected provider and referral entity 130 information (e.g., compare phone numbers, which may be obtained from the database 126) to determine that they are not the same, or re-select a provider and/or referral entity 130 as described above if they are the same.

The computing device 102 can provide a provider computing device 106 associated with the provider selected by the computing device 102 with access to the patient data 122 and the image data 124. Prior to providing the provider computing device 106 with access to the patient data 122 and the image data 124, the computing device 102 may generate a notification 132, such as an email, indicating that a case has been assigned to the provider associated with the provider computing device 106, but without any case-specific information, and send the notification 132 to the provider computing device 106. The provider, via the provider portal 120, may access the notification 132 and accept the case. Then the computing device 102 can provide the provider computing device 106 with secure access to the patient data 122 and the image data 124. For example, the computing device 102 may provide access via the provider portal 120 or a secure website for the provider to view the patient data 122 and the image data 124. Viewing such data via the provider portal 120 or secure website can allow the data to remain secure via the HIPAA-compliant server or database 126 where the data is stored. The provider may want to see more photographs of the spot on the patient's skin, so the provider computing device 106 can receive a selection from the provider to request more photos, and the computing device 102 can receive such request and automatically send a notification 132 to the patient computing device 104 indicating that more image data 124 should be uploaded.

The computing device 102 may receive case data 140 from the provider computing device 106. For example, the provider associated with the provider computing device 106 may be provided with access to the patient data 122, including the patient's medical history and symptoms, and the image data 124, including photographs of a spot on the patient's skin, for the provider to review and provide comments on, such as notes and a case disposition (e.g., benign, indeterminate, worrisome). Such information may be entered into the system 100 by the provider via the provider portal 120 and the information may be saved as case data 140 (e.g., in the database 126). The case data 140 can also include the provider's information, such as their name and credentials. In some embodiments, before receiving the case data 140, the computing device 102 may determine that more than a predetermined amount of time (e.g., 6 hours, 12 hours, 24 hours, or a time between 0-48 hours or longer) has passed since the patient data 122 and the image data 124 were received from the patient computing device 104 and that the case data 140 has not yet been received from the provider computing device 106. In response, computing device 102 may provide another provider computing device 106, which may be associated with a supervisory provider, with access to the patient data 122 and the image data 124 to provide the case data 140. In some examples, the computing device 102 may determine that the provider who missed the time deadline has a provider quality assurance rating below a predetermined threshold and set the provider on a probation status if the provider quality assurance rating is below the threshold, which may be noted in the provider's information in a database 126.

In some embodiments, the computing device 102 may include a quality assurance module 144. The quality assurance processor 144 can be configured to automatically retrieve a predetermined percentage of cases completed by providers (e.g., case data 140) for a quality assurance review. The quality assurance review can include the computing device 102 sending the case data 140 to a provider computing device 106 associated with a supervisor and/or providing a supervisor with access to the case data 140 (e.g., via the administration software 116) to ensure that the provider's case meets quality assurance standards set forth in the American Telemedicine Association guidelines or the American Academy of Dermatology guidelines, as non-limiting examples. In some examples, the computing device 102 may perform the quality assurance review.

The computing device 102 may generate a case summary report 142. For example, the administration software 116 can generate and store the case summary report 142. The case summary report 142 may be a file, such as a portable data format (PDF) file, a spreadsheet file (e.g., a comma-separated values (CSV) file, a Microsoft Excel file, etc.), a text file (e.g., a Microsoft Word file), or some other file format. The case summary report 142 can summarize the provider's impression of the patient's case into one of several easy-to-understand case dispositions, which may be color-coded. The case summary report 142 can be generated based at least in part on the patient data 122, the image data 124, and the case data 140. For instance, the case summary report 142 can include at least part of the patient data 122, such as the patient's biographical information (e.g., name, contact information, and date of birth, as non-limiting examples), at least part of the image data 124, such as one or more of the photographs of a spot on the patient's skin captured by the patient computing device 104, and at least part of the case data 140, such as the provider's information, comments and notes, and the case disposition (e.g., benign, indeterminate, or worrisome). When the case disposition is indeterminate or worrisome, the case summary report 142 can additionally include information about the referral entity 130, such as practice name, address, and phone number, as non-limiting examples. For example, the computing device 102 may parse the case data 140 for the case disposition and its value to determine whether to include the information about the referral entity 130.

The case summary report 142 can additionally include a summary of the case disposition. For instance, the computing device 102 may parse the case data 140 for the case disposition and its value to determine which summary and icon 208 to display. For example, if the case disposition is “benign,” the computer device 102 may include a summary such as “Appears benign at this time, no clinic visit necessary unless changes occur,” and a green icon 208 when generating the case summary report 142. If the case disposition is indeterminate, the computer device 102 may include a summary such as “Recommend clinic visit for evaluation. Although photo quality is acceptable, there is not a predominance of features to determine whether this is benign or worrisome. Recommend clinic visit for evaluation,” and a yellow icon 208 when generating the case summary report 142. If the case disposition is worrisome, the computer device 102 may include a summary such as “There are worrisome features demonstrated in this photograph. A clinic visit is recommended to evaluate,” and a red icon 208 when generating the case summary report 142.

In one aspect, to generate the case summary report 142, the computing device 102 may generate a color-coded disposition based on the case disposition in the case data 140 (e.g., generate the green, yellow, or red checkmark icon 208). The color coding can be generated by the computing device 102 (e.g., the administration software 116) to correlate with the provider's selected case disposition, where benign is green, indeterminate is yellow, and worrisome is red.

The computing device 102 can send the case summary report 142 to the referral entity 130 based on the case data 140. In particular, if the case data 140 indicates that the case disposition is indeterminate or worrisome, then the computing device 102 can securely send (e.g., via HIPPA-compliant servers) the case summary report 142 to the referral entity 130 for the patient to follow up with. For example, the computing device 102 may parse the case data 140 for the case disposition and/or the color-coded disposition and its value to determine whether to send the case summary report 142 to the referral entity 130. The case summary report 142 can be sent to the referral entity 130 within a predetermined amount of time (e.g., 12 hours, 24 hours, 48 hours, or a time between 0-48 hours or longer) after the computing device 102 received the patient data 122 and the image data 124 from the patient computing device 104. The case summary report 142 can also include the patient's contact information (e.g., from the patient data 122) for the referral entity 130 to be able to contact the patient. The case summary report 142 can be uploaded by the computing device 102 to the patient portal 118 for the patient, via the patient computing device 104, to be able to access and view the case summary report 142 via the administration software 116.

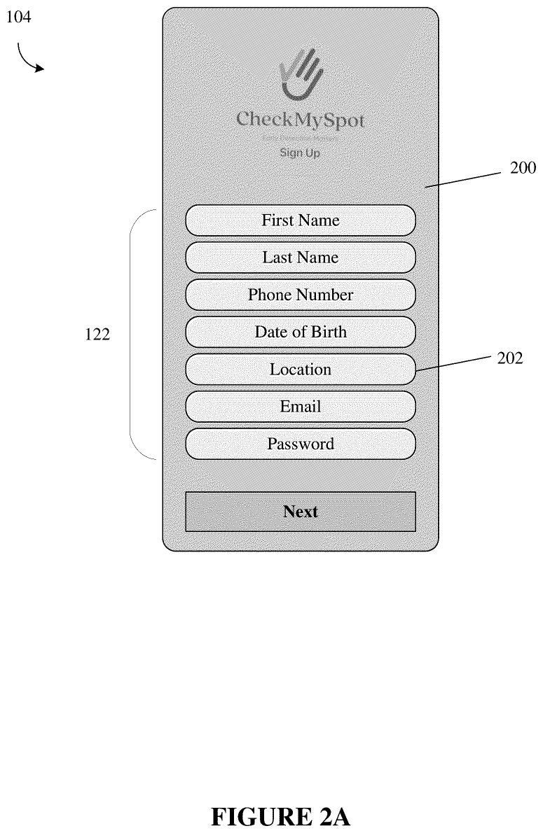

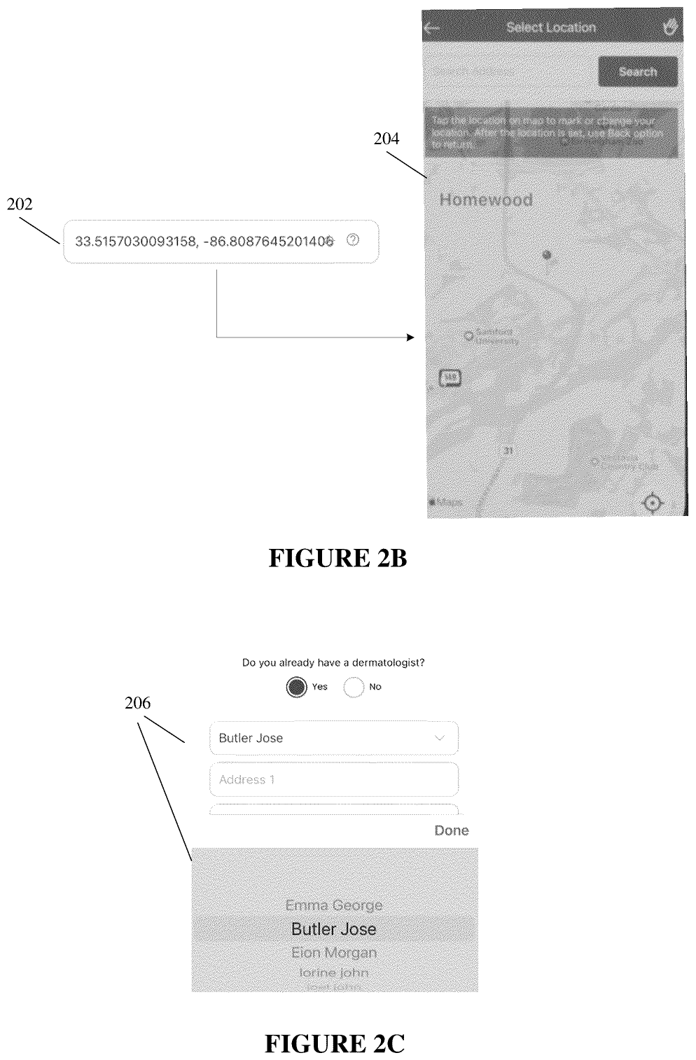

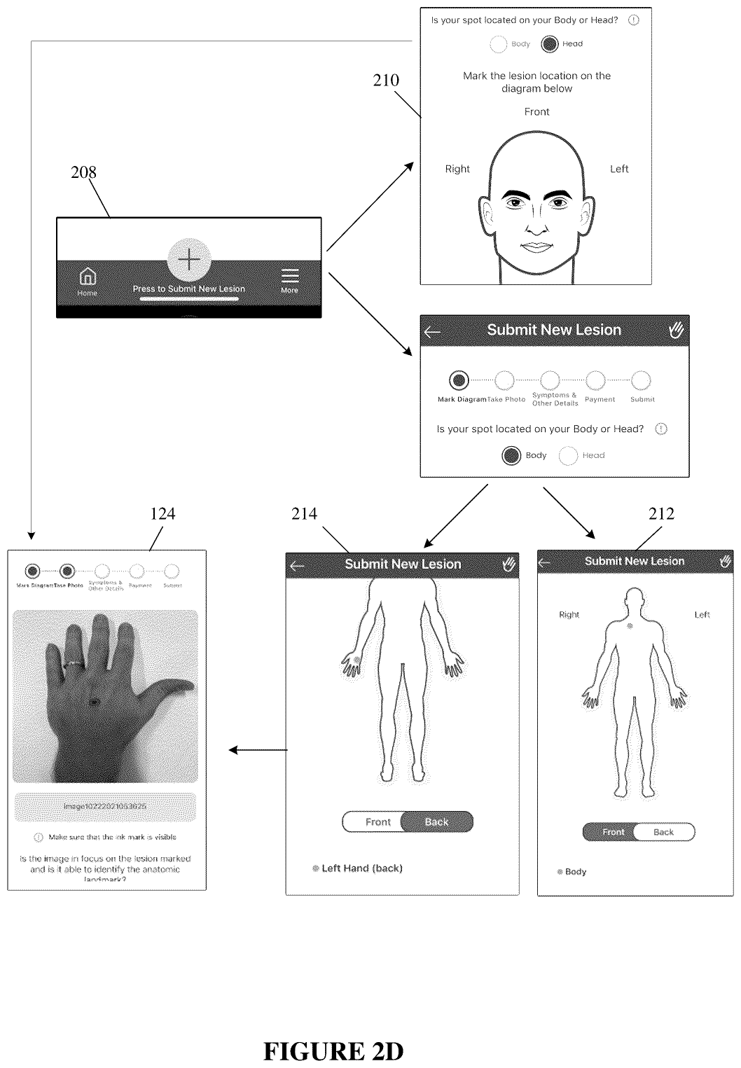

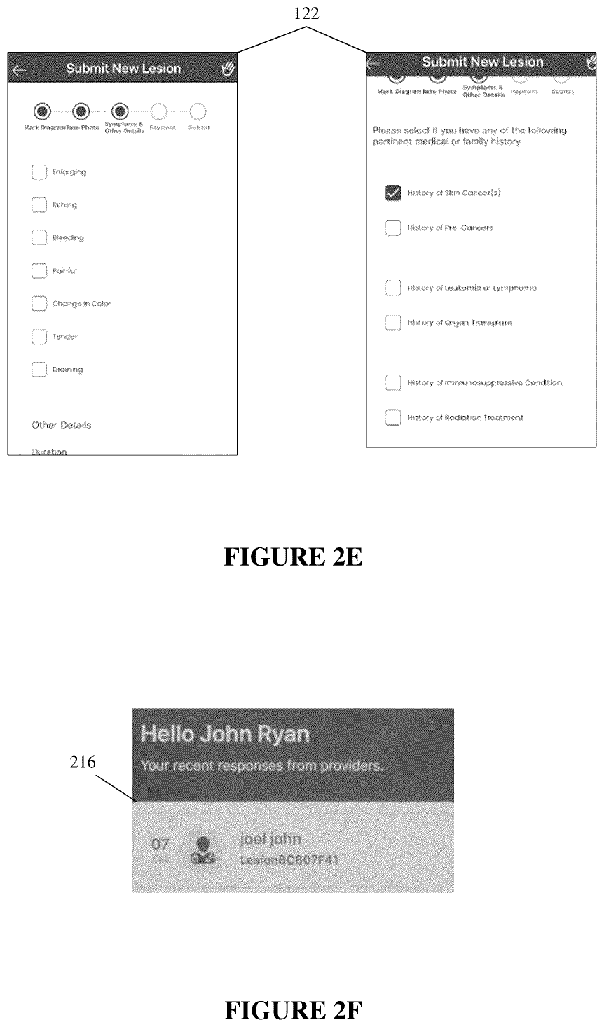

FIGS. 2A-2F are front views illustrating example embodiments of a patient interface 200 of a patient computing device 104 of the system 100 of the present disclosure. In the example of FIG. 2A, the patient interface 200 (e.g., the patient portal 118) of the patient computing device 104 may be an interface of a mobile application downloaded onto the patient computing device 104. The patient interface 200 can include an area (e.g., text boxes) for the patient to enter information to create an account, or to log into the patient's current account. The information the patient enters can be the patient data 122 (e.g., the patient's first name, last name, phone number, date of birth, email, and password). The information can also include the geolocation 202 of the patient computing device 104. The geolocation 202 can be determined by a geolocation algorithm that provides the coordinates of the patient computing device 104, as illustrated in FIG. 2B. In the example of FIG. 2B, a map 204 may also be displayed in the patient interface 200 of the patient computing device 104 to show the precise geolocation 202 of the patient computing device 104 as determined by the geolocation algorithms.

In the example of FIG. 2C, the patient may enter a preferred referral entity 130 (e.g., the patient's dermatologist). For example, the patient interface 200 may include a prompt generated by the computing device 102 (e.g., by the administration software 116) for the patient to select whether the patient has a preferred dermatologist for referral, and if “Yes” is selected, then the computing device 102 can generate a drop-down list 206 of qualified dermatologists for the patient to select from. Then, to assign the patient to the referral entity 130 based on the geolocation 202 of the patient computing device 104, the computing device 102 may determine that the patient selected a preferred referral entity 130 from the list 206 and assign the referral entity 130 as the preferred referral entity 130.

In the example of FIG. 2D, the patient may enter the image data 124 during a series of prompts generated by the computing device 102 (e.g., by the administration software 116) and displayed on the patient interface 200. For example, one of the prompts may be for the patient to select an icon 208 to upload one or more photos of a skin lesion to be examined by a medical provider. A next prompt may be for the patient to select whether the lesion is on the head 210 or body, and in response, the computing device 102 may generate a dynamic image of a head 210 or the front 212 or back 214 of the body (based on a patient selection) for the patient to select the area where the lesion is located on the head 210 or body. A next prompt can be generated that allows the patient to upload one or more photographs of a spot on the patient's skin taken by the patient computing device 104. The photographs of the spot on the patient's skin may be uploaded as the image data 124 to be received by the computing device 102. The photographs of the spot on the patient's skin may be uploaded, for example, when the patient selects a button on the patient interface 200 to submit the photographs.

In the example of FIG. 2E, the patient may enter the patient data 122, which may include information about the patient's symptoms and medical history. For example, the patient interface 200 may include prompts (e.g., checkboxes, dropdowns, textboxes, and/or other items for inputting information) generated by the computing device 102 (e.g., by the administration software 116) for the patient to select the symptoms and medical history, and to save the responses (e.g., by selecting a button). The patient's selection of one or more of the prompts can then be received by the computing device 102 as the patient data 122, such as when the patient selects a button on the patient interface 200 to submit the patient data 122.

FIG. 2F illustrates a view of the patient interface 200 when a case summary report 142 has been generated to notify the patient that the patient's case has been sent to and reviewed by a medical provider via the system 100. For example, the computing device 102 may send a notification 132 to the patient computing device 104 and the patient portal 118 can be accessed by the patient entering login credentials to view the case summary report 142. In some embodiments, the patient portal 118 may also include access to prior case summary reports 142 and previous photographs of spots on the patient's skin, for example so the patient can track any changes in a skin lesion.

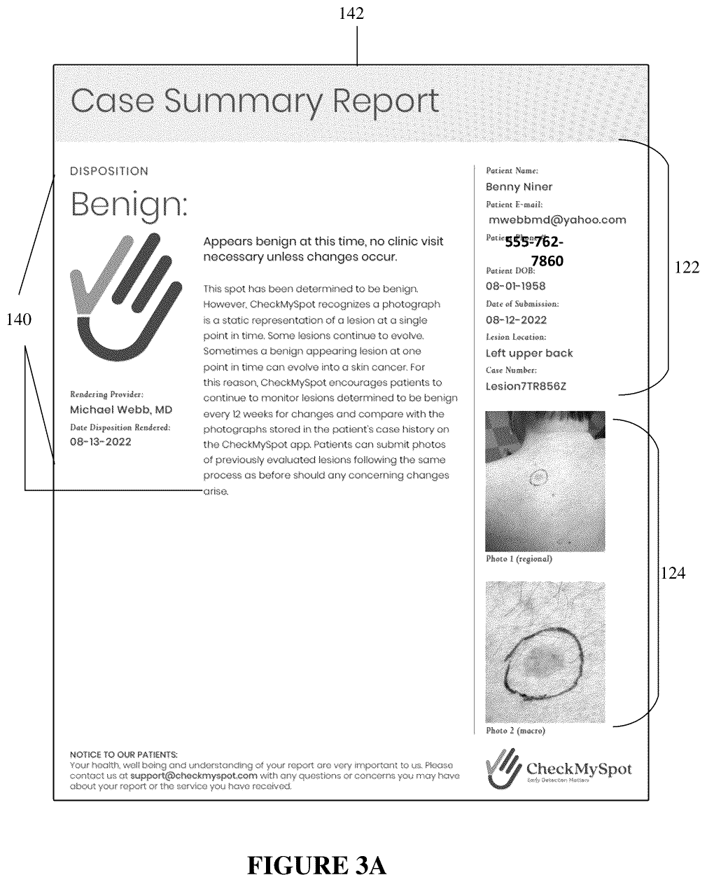

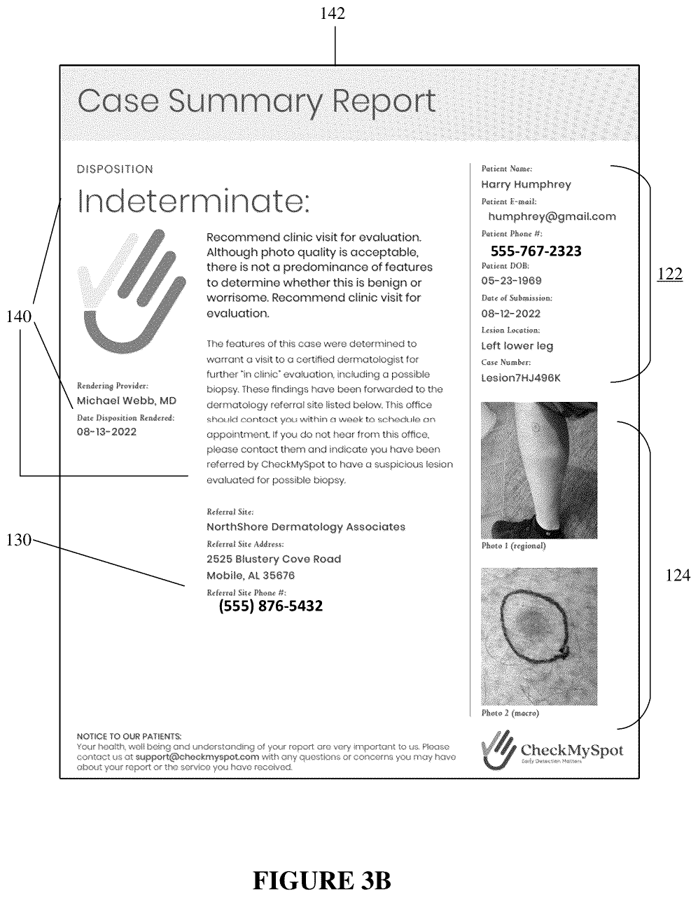

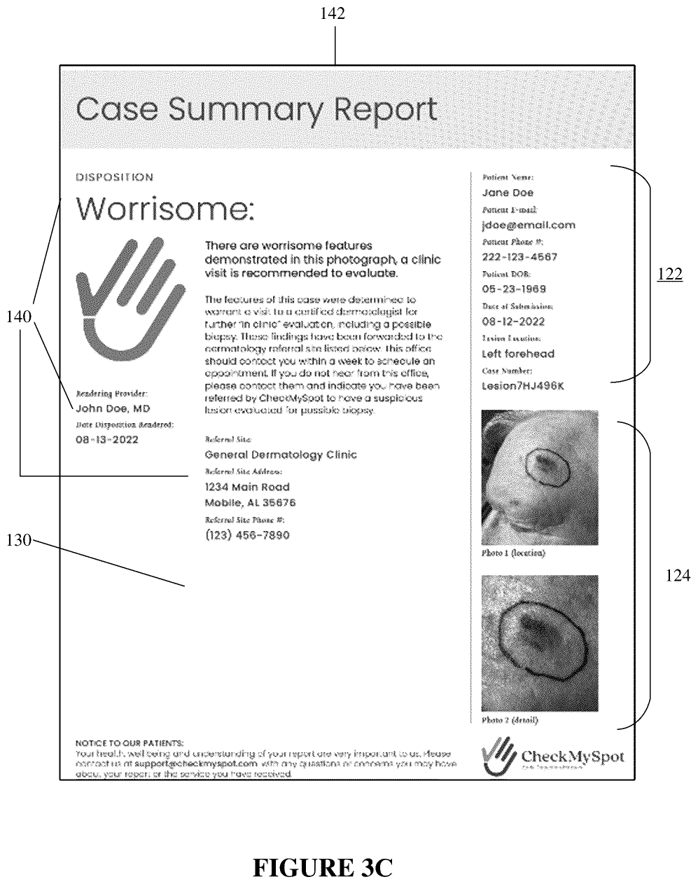

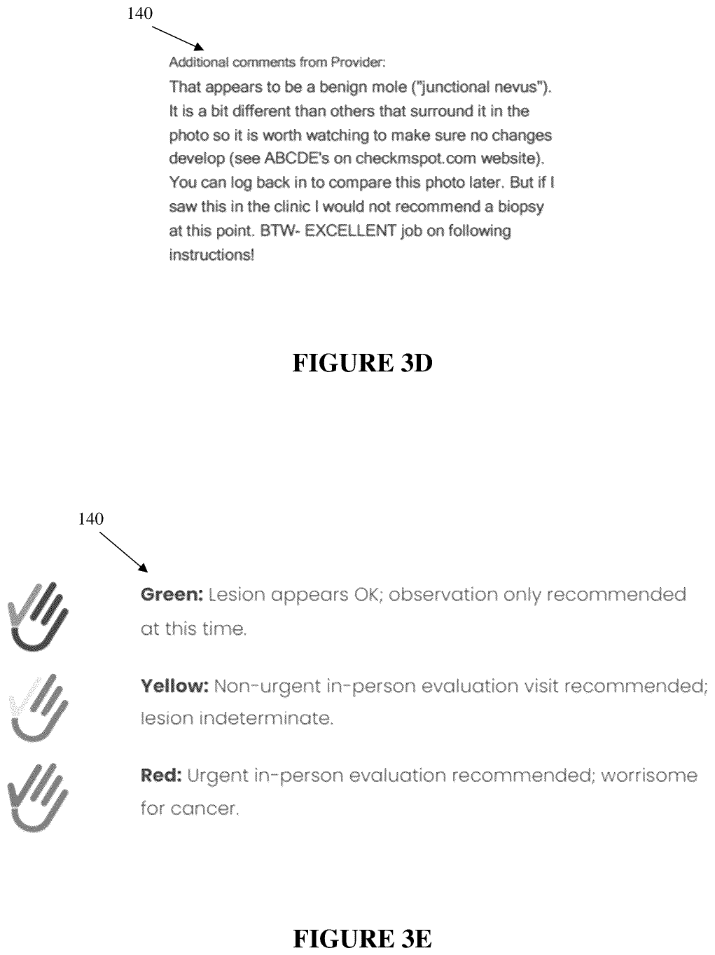

FIGS. 3A-3E are front views illustrating example embodiments of case summary reports 142 generated by the system 100. In particular, FIGS. 3A-3C are illustrations of example case summary reports 142. In FIG. 3A, an example case summary report 142 when the case disposition provided by the provider is “benign” is illustrated. The case summary report 142 can include at least part of the patient data 122, the image data 124, and the case data 140. For example, the patient data 122 included in the case summary report 142 may be the patient's name, e-mail, phone number, date of birth, and location of the lesion, which may have been entered through the patient interface 200 (e.g., the patient portal 118) of the patient computing device 104 and received by the computing device 102. At least part of the image data 124 comprising the photographs of a spot on the patient's skin captured by the patient computing device 104 and received by the computing device 102 can be included in the case summary report 142. For example, the patient computing device 104 may have captured more than one photograph of a spot on the patient's skin and the case summary report 142 may include one or several of the photographs, such as a regional view and a macro view. At least part of the case data 140, such as the case disposition, provider name, and provider notes, can be included in the case summary report 142. Additionally, the case summary report 142 can include the date the patient submitted the patient data 122, the image data 124, and the date the provider submitted the case data 140. A case number generated by the computing device 102 may also be included in the case summary report 142. The case data 140 can also include text generated by the computing device 102 based on the case disposition. For example, when the case disposition is “benign,” text outlining a follow-up procedure and an explanation of a benign-appearing lesion may be generated by the computing device 102 and added to the case summary report 142 when the computing device 102 generates the report.

In FIG. 3B, an example case summary report 142 when the case disposition provided by the provider is “indeterminate” is illustrated. Similar to the case summary report 142 illustrated in FIG. 3A, at least part of the patient data 122, the image data 124, and the case data 140, as well as the date the patient submitted the patient data 122 and the image data 124 and the date the provider submitted the case data 140, can be included in the report. When the case disposition is “indeterminate,” the case summary report 142 may additionally include contact information for the referral entity 130, such as the referral entity's 130 name, address, and phone number. The computing device 102 may also send the case summary report 142 to the referral entity 130. The case data 140 can also include text generated by the computing device 102 based on the case disposition. For example, when the case disposition is “indeterminate,” text explaining that a clinic visit is recommended may be generated by the computing device 102 and added to the case summary report 142 when the computing device 102 generates the report.

In FIG. 3C, an example case summary report 142 when the case disposition provided by the provider is “worrisome” is illustrated. Similar to the case summary report 142 illustrated in FIG. 3A, at least part of the patient data 122, the image data 124, and the case data 140, as well as the date the patient submitted the patient data 122 and the image data 124 and the date the provider submitted the case data 140, can be included in the report. When the case disposition is “worrisome,” the case summary report 142 may additionally include contact information for the referral entity 130, such as the referral entity's 130 name, address, and phone number. The computing device 102 may also send the case summary report 142 to the referral entity 130. The case data 140 can also include text generated by the computing device 102 based on the case disposition. For example, when the case disposition is “worrisome,” text explaining that a clinic visit is recommended may be generated by the computing device 102 and added to the case summary report 142 when the computing device 102 generates the report.

FIG. 3D illustrates additional case data 140 that may be included in the case summary report 142. For example, the provider may include additional notes when evaluating the patient data 122 and the image data 124 and enter the additional notes using the provider computing device 106 (e.g., via the provider portal 120), which can be collected by the computing device 102 to be included in the case summary report 142 when the report is generated by the computing device 102. FIG. 3E illustrates exemplary case dispositions categorized into three categories, however any number of case disposition categories may be contemplated. In the example of FIG. 3E, three case dispositions are provided: benign (green), indeterminate (yellow), and worrisome (red), each of which is embodied in a checkmark graphic. These example case dispositions may be included in the case data 140 and added to the case summary report 142 by the computing device 102 when generating the report. For instance, both the graphic and the text describing the case disposition may be included in the case summary report 142.

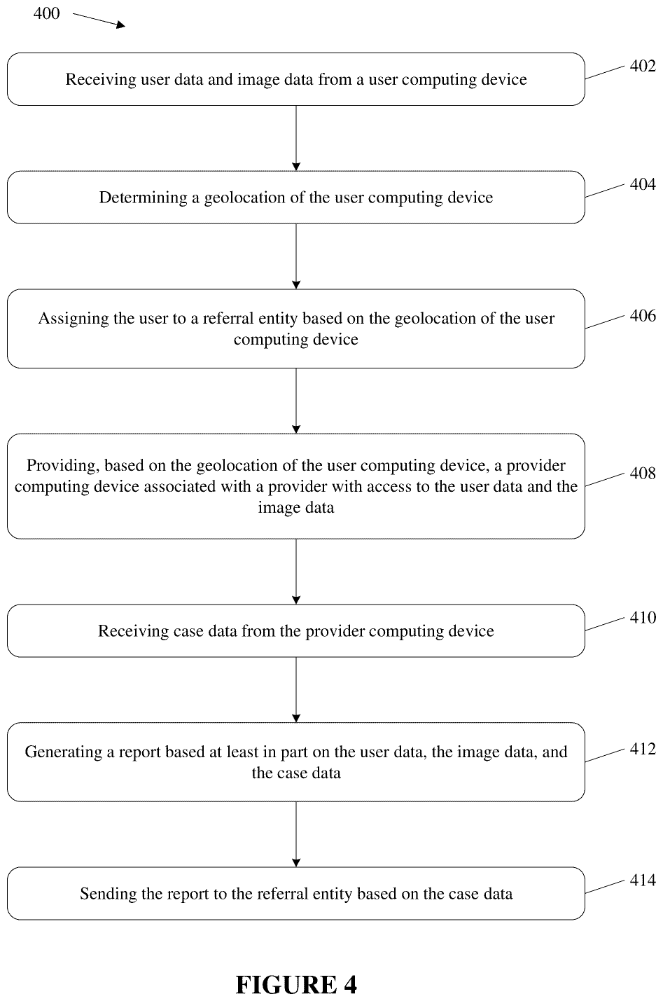

FIG. 4 is a flowchart diagram illustrating an example embodiment of a computer-implemented method 400 of the present disclosure. In certain embodiments, the computer-implemented method 400 of a telemedicine platform for location-based provider screening and referral may include the step of providing one or more memories 110. The memories 110 may store non-transitory computer-executable instructions 112 for the telemedicine platform for location-based provider screening and referral. The computer-implemented method 400 may include the step of providing a computing device 102 with at least one processor 108 for executing the non-transitory computer-executable instructions 112. The computer-implemented method 400 may include the step of executing, by the computing device 102, the non-transitory computer-executable instructions 112 to perform operations.

The computer-implemented method 400 may include an operation 402 of receiving, by a computing device 102, patient data 122, and image data 124 from a patient computing device 104 associated with a patient. The patient data 122 may comprise one or more of biographical information, medical history, or symptoms of the patient associated with the patient computing device 104. The image data 124 may comprise one or more photographs of a spot on the patient's skin captured by a camera of the patient computing device 104.

The computer-implemented method 400 may include an operation 404 of determining, by the computing device 102, a geolocation 202 of the patient computing device 104.

The computer-implemented method 400 may include an operation 406 of assigning, by the computing device 102, the patient to a referral entity 130 based on the geolocation 202 of the patient computing device 104. In some examples, the computer-implemented method 400 may assign the patient to the referral entity 130 by identifying, based on the geolocation 202 of the patient computing device 104, one or more referral entities 130 located within a predetermined distance from the geolocation 202 of the patient computing device 104, receiving a rating associated with each of the one or more referral entities 130, and selecting the referral entity 130 based on the rating associated with the referral entity 130, wherein the selected referral entity 130 is the highest rated of the referral entities 130 based on the referral entity ratings 134. In some examples, the computer-implemented method 400 may assign the patient to the referral entity 130 by identifying, based on the geolocation 202 of the patient computing device 104, one or more referral entities 130 located within a predetermined distance from the geolocation 202 of the patient computing device 104, determining that a preferred referral entity 130 selected by the patient is one of the one or more referral entities 130, and obtaining, from a database 126 configured to store information associated with the one or more referral entities 130, information associated with the preferred referral entity 130, wherein sending the report 141 to the referral entity 130 comprises sending the report 142 to the preferred referral entity 130. In some examples, the computer-implemented method 400 may assign the patient to the referral entity 130 by identifying, based on the geolocation 202 of the patient computing device 104, one or more referral entities 130 located within a predetermined distance from the geolocation 202 of the patient computing device 104, determining that a preferred referral entity 130 selected by the patient is one of the one or more referral entities 130, determining that the preferred referral entity 130 is not in a database 126 configured to store information associated with the one or more referral entities 130, and adding an entry to the database 126, the entry comprising information associated with the preferred referral entity 130, wherein sending the report 142 to the referral entity 130 comprises sending the report 142 to the preferred referral entity 130. In some examples, the referral entity 130 is selected by the patient.

The computer-implemented method 400 may include an operation 408 of providing, by the computing device 102 based on the geolocation 202 of the patient computing device 104, a provider computing device 106 associated with a provider with access to the patient data 122 and the image data 124. In some examples, the provider is different from the referral entity 130.

The computer-implemented method 400 may include an operation 410 of receiving, by the computing device 102, case data 140 from the provider computing device 106. The case data 140 may comprise one or more of provider information associated with the provider, comments from the provider, or a case disposition.

The computer-implemented method 400 may include an operation 412 of generating, by the computing device 102, a report 142 based at least in part on the patient data 122, the image data 124, and the case data 140. In some examples, the computer-implemented method 400 may generate the report 142 by generating a color-coded disposition based on a case disposition included in the case data 140, creating the report 142 based on at least in part on the patient data 122, the image data 124, and the case data 140, wherein the report 142 includes at least a portion of the patient data 122, the image data 124, and the case data 140, and determining whether to send the report 142 to the referral entity 130 based on the color-coded disposition.

The computer-implemented method 400 may include an operation 414 of sending, by the computing device 102, the report 142 to the referral entity 130 based on the case data 140. In some examples, the report 142 is sent to the referral entity 130 within a predetermined amount of time after the patient data 122 and the image data 124 are received from the patient computing device 104.

In some embodiments, the computer-implemented method 400 may further include an operation of, prior to receiving the case data 140 from the provider computing device 106, determining that more than a predetermined amount of time has passed since the patient data 122 and the image data 124 were received from the patient computing device 104, determining that the case data 140 has not been received from the provider computing device 106, and providing a second provider computing device 106 associated with a supervisory provider with access to the patient data 122 and the image data 124. In some examples, the computer-implemented method 400 may further include an operation of determining that the provider has a provider quality assurance rating below a predetermined threshold and setting the provider on a probation status.

In some embodiments, the computer-implemented method 400 may further include an operation of, prior to providing the provider computing device 106 with access to the patient data 122 and the image data 124, generate a notification 132 indicating that a case has been assigned to the provider, and sending the notification 132 to the provider computing device 106.

In some embodiments, the computer-implemented method 400 may further include an operation of, prior to providing the provider computing device 106 with access to the patient data 122 and the image data 124, receiving a schedule 136 from each of a plurality of provider computing devices 106, each provider computing device 106 associated with a provider of a plurality of providers, wherein each respective schedule 136 identifies an availability of a provider associated with the respective schedule 136, receiving a provider rating 138 associated with each of the plurality of providers, and selecting the provider based on the rating associated with the provider, wherein the selected provider is the highest rated of the plurality of providers based on the provider ratings 138 and is available based on the availability of the provider. In some examples, the rating associated with each of the plurality of providers comprises a provider quality assurance rating for each provider of the plurality of providers, wherein the provider quality assurance rating is based on the timeliness of a provider associated with the provider quality assurance rating. In some examples, the rating associated with each of the plurality of providers comprises a provider quality assurance rating for each provider of the plurality of providers, wherein the provider quality assurance rating is further based on the performance of a provider associated with the provider quality assurance rating in a quality assurance review.

In some embodiments, the computer-implemented method 400 may further include an operation of sending the patient data 122 and the image data 124 to a database 126 configured to store the patient data 122 and the image data 124.

The presently disclosed systems and methods have a wide application anywhere in the computer and healthcare industries where a telemedicine platform is needed. One particularly important application for the systems and methods described herein relates to asynchronous tele-dermatology. However, the systems and methods described above could be utilized in other contexts.

Those skilled in the art will recognize improvements and modifications to the preferred embodiments of the present disclosure. All such improvements and modifications are considered within the scope of the concepts disclosed herein and the claims that follow.

As used herein, the term “computing device” may include a processor-controlled device, such as, by way of example, a personal computer, workstation, server, client, mini-computer, mainframe computer, desktop computer, laptop computer, smartphone, tablet, network of one or more individual computers, mobile computer, portable computer, handheld computer, or any combination thereof. The described systems and techniques may be performed by a system that includes a single computing device or more than one computing device unless otherwise specified.

A computing device may include an integrated circuit (IC) and may include an application-specific integrated circuit (ASIC) or some other type of IC. A computing device may be a uniprocessor or multiprocessor machine. Accordingly, a computing device may include one or more processors and, thus, the system may also include one or more processors. Examples of processors include sequential state machines, microprocessors, microcontrollers, graphics processing units (GPUs), central processing units (CPUs), application processors, digital signal processors (DSPs), reduced instruction set computing (RISC) processors, systems on a chip (SoC), baseband processors, field-programmable gate arrays (FPGAs), programmable logic devices (PLDs), gated logic, and other suitable hardware configured to perform the various functionality described throughout this disclosure. In some embodiments, features of the system can be implemented primarily in hardware using, for example, hardware components such as application-specific integrated circuits (ASICs) or field-programmable gated arrays (FPGAs). Implementation of the hardware circuitry will be apparent to persons skilled in the relevant art(s). In yet another embodiment, features of the system can be implemented using a combination of both general-purpose hardware and software.

The computing device may include one or more memories. Accordingly, the system may include one or more memories. A memory may include a memory storage device or an addressable storage medium which may include, by way of example, random access memory (RAM), static random access memory (SRAM), dynamic random access memory (DRAM), electronically erasable programmable read-only memory (EEPROM), programmable read-only memory (PROM), erasable programmable read-only memory (EPROM), hard disks, floppy disks, laser disk players, digital video disks, compact disks, videotapes, audio tapes, magnetic recording tracks, magnetic tunnel junction (MTJ) memory, optical memory storage, quantum mechanical storage, electronic networks, and/or other devices or technologies used to store electronic content such as programs and data. A basic input/output system (BIOS) can include basic routines that help to transfer information between elements within the system, such as during start-up, can be stored in the one or more memories.

The system can also include one or more storage devices. Examples of a storage device include, but are not limited to, a hard disk drive, a magnetic disk drive, an optical disc drive in combination with an optical medium, a solid-state memory device, or any combination thereof. A storage device can be connected to a bus by an appropriate interface, such as an SCSI, advanced technology attachment (ATA), serial ATA, universal serial bus (USB), IEEE 1394 (FIREWIRE), or any combination thereof. A storage device (or one or more components thereof) can be removably interfaced with the system (e.g., via an external port connector). The storage device and an associated computer-readable medium can provide nonvolatile and/or volatile storage of computer-executable instructions, data structures, program modules, and/or other data for the system.

In particular, the one or more memories may store computer-executable instructions that, when executed by the one or more processors, cause the one or more processors to implement the procedures and techniques described herein. The one or more processors may be operably associated with the one or more memories so that the computer-executable instructions can be provided to the one or more processors for execution. For example, the one or more processors may be operably associated to the one or more memories through one or more buses. Furthermore, the computing device may possess or may be operably associated with input devices (e.g., a keyboard, a keypad, controller, a mouse, a microphone, a touch screen, a sensor) and output devices such as (e.g., a computer screen, printer, or a speaker).

The computer-executable instructions described herein can be downloaded to respective computing/processor devices from a computer-readable storage medium or to an external computer or external storage device via a network, for example, the Internet, a local area network (LAN), a wide area network (WAN) and/or a wireless network. The network may comprise copper transmission cables, optical transmission fibers, wireless transmission, routers, firewalls, switches, gateway computers and/or edge servers. A network adapter card or network interface in each computing/processor device receives computer readable program instructions from the network and forwards the computer readable program instructions for storage in a computer readable storage medium within the respective computing/processor device.

The computing device may execute an appropriate operating system such as LINUX®, UNIX®, MICROSOFT® WINDOWS®, APPLE® MACOS®, IBM® OS/2®, ANDROID, and/or the like. The computing device may advantageously be equipped with a network communication device such as a network interface card, a modem, or other network connection device suitable for connecting to one or more networks.

A computing device may advantageously contain control logic, or program logic, or other substrate configuration representing data and instructions, which cause the computing device to operate in a specific and predefined manner as, described herein. In particular, the computing device programs, when executed, enable a control processor to perform and/or cause the performance of features or operations of the present disclosure. The control logic may advantageously be implemented as one or more modules. The modules may advantageously be configured to reside on the computing device memory and execute on the one or more processors. The modules include, but are not limited to, software or hardware components that perform certain tasks. Thus, a module may include, by way of example, components, such as, software components, processes, functions, subroutines, procedures, attributes, class components, task components, object-oriented software components, segments of program code, drivers, firmware, micro-code, circuitry, data, and/or the like. The control logic conventionally includes the manipulation of digital bits by the processor and the maintenance of these bits within memory storage devices resident in one or more of the memory storage devices. Such memory storage devices may impose a physical organization upon the collection of stored data bits, which are generally stored by specific electrical or magnetic storage cells. The control logic generally performs a sequence of computer-executed steps. These steps generally require manipulations of physical quantities. Usually, although not necessarily, these quantities take the form of electrical, magnetic, or optical signals capable of being stored, transferred, combined, compared, or otherwise manipulated. It is conventional for those skilled in the art to refer to these signals as bits, values, elements, symbols, characters, text, terms, numbers, files, or the like. It should be kept in mind, however, that these and some other terms should be associated with appropriate physical quantities for computer operations, and that these terms are merely conventional labels applied to physical quantities that exist within and during operation of the computer based on designed relationships between these physical quantities and the symbolic values they represent.

As will be appreciated by one skilled in the art, aspects of the present disclosure may be embodied as an apparatus, system, method, computer program product, or the like. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present disclosure may take the form of a computer program product embodied in one or more computer readable medium(s) having program code embodied thereon.

It should be understood that manipulations within the computing device are often referred to in terms of adding, comparing, moving, searching, or the like, which are often associated with manual operations performed by a human operator. It is to be understood that no involvement of the human operator may be necessary, or even desirable. The operations described herein are machine operations performed in conjunction with the human operator or user that interacts with the computing device or computing devices.

It should also be understood that the programs, modules, processes, methods, and the like, described herein are but an exemplary implementation and are not related, or limited, to any particular computing device, apparatus, or computer language. Rather, various types of general-purpose computing machines or devices may be used with programs constructed in accordance with some of the teachings described herein. In some embodiments, very specific computing machines, with specific functionality, may be required. Similarly, it may prove advantageous to construct a specialized apparatus to perform the method steps described herein by way of dedicated systems with hard-wired logic or programs stored in nonvolatile memory, such as, by way of example, read-only memory (ROM).

Aspects of the present disclosure are described herein with reference to flowchart illustrations or block diagrams of methods, apparatuses, systems, or computer program products according to embodiments of the disclosure. It will be understood that each block of the flowchart illustrations and/or block diagrams, and combinations of blocks in the flowchart illustrations and/or block diagrams, can be implemented by computer-executable instructions. These computer-executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-executable instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The schematic flow chart diagrams included herein are generally set forth as logical flow chart diagrams. As such, the depicted order and labeled steps are indicative of one embodiment of the presented method. Other steps and methods may be conceived that may be equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown. The schematic flowchart diagrams and/or schematic block diagrams in the Figures illustrate the architecture, functionality, and operation of possible implementations of apparatuses, systems, methods and computer program products according to various embodiments of the present disclosure. In this regard, each block in the schematic flowchart diagrams and/or schematic block diagrams may represent a module, segment, or portion of code, which comprises one or more executable instructions of the program code for implementing the specified logical function(s).

While the making and using of various embodiments of the present disclosure are discussed in detail herein, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those skilled in the art will recognize, or be able to ascertain, using no more than routine experimentation, numerous equivalents to the specific substances and procedures described herein. Such equivalents are considered to be within the scope of this disclosure and are covered by the following exemplary claims.

Furthermore, the described features, structures, or characteristics of the disclosure may be combined in any suitable manner in one or more embodiments. In the description contained herein, numerous specific details are provided to provide understanding of embodiments of the disclosure. One skilled in the relevant art will recognize, however, that the disclosure may be practiced without one or more of the specific details, or with other methods, components, materials, apparatuses, devices, systems, and so forth. In other instances, well-known structures, materials, or operations may not be shown or described in detail to avoid obscuring aspects of the disclosure.

What is claimed is:

1. A computer-implemented method for a telemedicine platform for location-based provider screening and referral, comprising:
receiving, by a computing device, patient data and image data from a patient computing device associated with a patient;
determining, by the computing device, a geolocation of the patient computing device;
assigning, by the computing device, the patient to a referral entity based on the geolocation of the patient computing device;
providing, by the computing device based on the geolocation of the patient computing device, a first provider computing device associated with a first provider with access to the patient data and the image data;
receiving, by the computing device, case data from the first provider computing device;
generating, by the computing device, a report based at least in part on the patient data, the image data, and the case data; and
sending, by the computing device, the report to the referral entity based on the case data.

2. The computer-implemented method of claim 1, further comprising:
prior to receiving the case data from the first provider computing device, determining that more than a predetermined amount of time has passed since the patient data and the image data were received from the patient computing device;
determining that the case data has not been received from the first provider computing device; and
providing a second provider computing device associated with a supervisory provider with access to the patient data and the image data.

3. The computer-implemented method of claim 2, further comprising:
determining that the first provider has a provider quality assurance rating below a predetermined threshold; and
setting the first provider on a probation status.

4. The computer-implemented method of claim 1, further comprising:
generating a notification indicating that a case has been assigned to the first provider; and
sending the notification to the first provider computing device.

5. The computer-implemented method of claim 1, wherein assigning the patient to the referral entity based on the geolocation of the patient computing device comprises:
identifying, based on the geolocation of the patient computing device, one or more referral entities located within a predetermined distance from the geolocation;
receiving one or more referral entity ratings each associated with one of the one or more referral entities; and
selecting the referral entity based on the referral entity rating associated with the referral entity, wherein the selected referral entity is the highest rated of the one or more referral entities based on the referral entity ratings.

6. The computer-implemented method of claim 1, wherein assigning the patient to the referral entity based on the geolocation of the patient computing device comprises:
identifying, based on the geolocation of the patient computing device, one or more referral entities located within a predetermined distance from the geolocation;
determining that a preferred referral entity selected by the patient is one of the one or more referral entities; and
obtaining, from a database configured to store information associated with the one or more referral entities, information associated with the preferred referral entity;
wherein sending the report to the referral entity comprises sending the report to the preferred referral entity.

7. The computer-implemented method of claim 1, wherein assigning the patient to the referral entity based on the geolocation of the patient computing device comprises:
identifying, based on the geolocation of the patient computing device, one or more referral entities located within a predetermined distance from the geolocation;
determining that a preferred referral entity selected by the patient is one of the one or more referral entities;
determining that the preferred referral entity is not in a database configured to store information associated with the one or more referral entities; and
adding an entry to the database, the entry comprising information associated with the preferred referral entity;
wherein sending the report to the referral entity comprises sending the report to the preferred referral entity.

8. The computer-implemented method of claim 1, further comprising:
prior to providing the first provider computing device with access to the patient data and the image data,
receiving a schedule from each of a plurality of provider computing devices including the first provider computing device, each provider computing device associated with a provider of a plurality of providers, wherein each respective schedule identifies an availability of a provider associated with the respective schedule;
receiving one or more provider ratings each associated with one of the plurality of providers; and
selecting the first provider from the plurality of providers based on the provider ratings associated with the plurality of providers, wherein the selected provider is the highest rated of the plurality of providers based on the provider ratings and is available based on the availability of the provider.

9. The computer-implemented method of claim 8, wherein the provider rating associated with each of the plurality of providers comprises a provider quality assurance rating for each provider of the plurality of providers, wherein the provider quality assurance rating is based on timeliness of a provider associated with the provider quality assurance rating.

10. The computer-implemented method of claim 8, wherein the rating associated with each of the plurality of providers comprises a provider quality assurance rating for each provider of the plurality of providers, wherein the provider quality assurance rating is further based on an adherence of a provider associated with the provider quality assurance rating to a quality assurance review.

11. The computer-implemented method of claim 1, wherein generating the report based at least in part on the patient data, the image data, and the case data comprises:
generating a color-coded disposition based on a case disposition included in the case data;
creating the report based at least in part on the patient data, the image data, and the case data, wherein the report includes at least a portion of the patient data, the image data, and the case data; and
determining whether to send the report to the referral entity based on the color-coded disposition.

12. The computer-implemented method of claim 1, wherein the report is sent to the referral entity within a predetermined amount of time after the patient data and the image data are received from the patient computing device.

13. The computer-implemented method of claim 1, wherein the patient data comprises one or more of biographical information, medical history, or symptoms of the patient associated with the patient computing device.

14. The computer-implemented method of claim 1, wherein the image data comprises one or more photographs of a spot on the patient's skin captured by a camera of the patient computing device.

15. The computer-implemented method of claim 1, wherein the case data comprises one or more of provider information associated with the provider, comments from the provider, or a case disposition.

16. The computer-implemented method of claim 1, further comprising:
sending the patient data and the image data to a database configured to store the patient data and the image data.

17. The computer-implemented method of claim 1, wherein the referral entity is selected by the patient.

18. The computer-implemented method of claim 1, wherein the provider is different from the referral entity.

19. A non-transitory computer-readable storage medium having executable instructions stored thereon, wherein the executable instructions, when executed by a processor, are configured to:
receive patient data and image data from a patient computing device associated with a patient;
determine a geolocation of the patient computing device;
assign the patient to a referral entity based on the geolocation of the patient computing device;
provide, based on the geolocation of the patient computing device, a provider computing device associated with a provider with access to the patient data and the image data;
receive case data from the provider computing device;
generate a report based at least in part on the patient data, the image data, and the case data; and
send the report to the referral entity based on the case data.

20. A system for a telemedicine platform for location-based provider screening and referral, comprising:
at least one memory storing non-transitory computer-executable instructions;
a computing device with one or more processors for executing the non-transitory computer-executable instructions;
a patient computing device with one or more input devices and associated with a patient; and
a provider computing device associated with a provider;
wherein, when executed by the computing device, the non-transitory computer-executable instructions cause the one or more processors of the computing device to perform operations comprising:
receiving patient data and image data from the patient computing device;
determining a geolocation of the patient computing device;
assigning the patient to a referral entity based on the geolocation of the patient computing device;
providing, based on the geolocation of the patient computing device, the provider computing device with access to the patient data and the image data;
receiving case data from the provider computing device;
generating a report based at least in part on the patient data, the image data, and the case data; and
sending the report to the referral entity based on the case data.
